(19) United States
(12) Patent Application Publication
(10) Pub. No.: US 2002/0177790 A1
(43) Pub. Date: Nov. 28, 2002

(54) Device and method for establishingstability in an implant or unit

(76) Inventors: Neil Meredith, Leeds (GB); Anders Petersson, Goteborg (SE)

Correspondence Address: Synnestvedt & Lechner LLP, 2600 Aramark Tower, 1101 Market Street, Philadelphia, PA, 19107-2950, US

(21) Appl. No.: 10/100,392

(22) Filed: Mar. 18, 2002

(30) Foreign Application Priority Data
Sep. 16, 1999 (SE) 9903304-5
Sep. 12, 2000 (WO) WO 01/19248 A1

(51) Int. Cl. A61B 5/103

(52) U.S. Cl. 600/587

(57) ABSTRACT

Device for determining the stability of an implant fitted in the human body, where a vibration-producing unit cooperates with the implant and gives a frequency signal (i1) which is a function of its stability and characteristic features, characterized in that it operates with a digital information signal (i2) which represents the stability and feature and is generated in a receiver unit which receivers the frequency signal (i1) and converts it to the digital information signal (i2), in that the information item(s) representing the characteristic features are included in or connected to information-containing elements (memories) included in or connected to the device, and in that one or more information signals (i4) principally represent only the stability, which information signal or signals (i4) result(s) from the processing, in an arithmetic unit, belonging to or connected to the device, of the digital information signal (i2) and the information item(s) representing the characteristic features in order to permit information relating only to the stability to be displayed in a display unit.

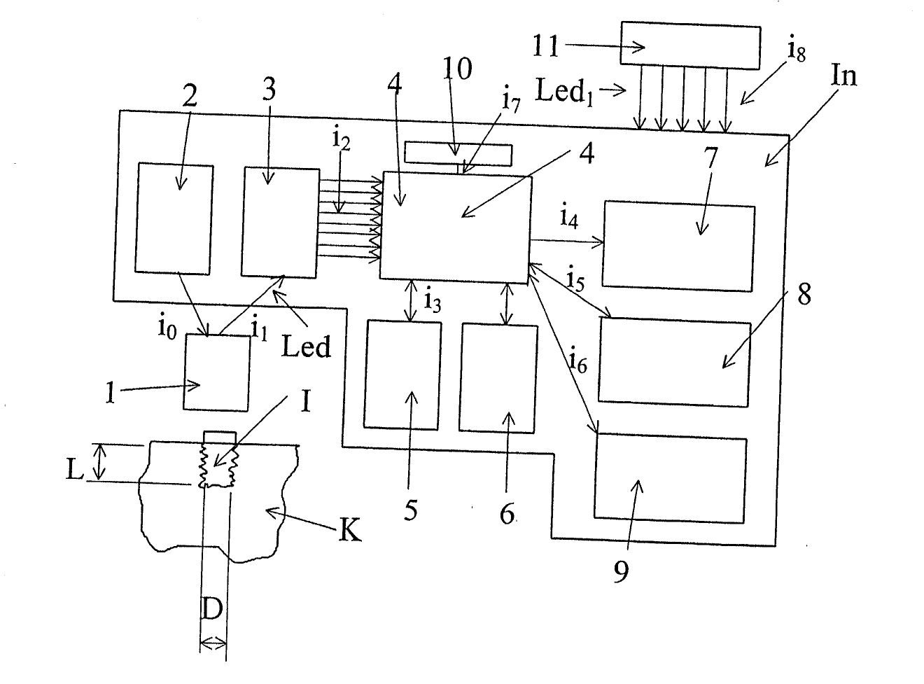

The invention relates to a device and method for the measurement of implant stability as a function of, amongst other things, the resonance frequency of the implant, of the implant with an attached structure and/or of a transducer in contact with the implant or a structure that is attached to the implant. The invention also relates to establish the stability of an anchorage of a first unit. It also relates to the preamble of claims 1, 2, 3, 9 and 12. Bone anchored threaded any cylindrical metallic, endosseous implants are now widely used in Medicine and Dentistry. Such implants are inserted into a pre-drilled hole in the facial skeleton and used to provide a means of anchorage for a dental or facial prosthesis which may be a single replacement tooth, a bridge, a denture or even a false eye or ear. Implants may be placed as a one or two stage procedure. In a one stage procedure the implant is placed and exposed immediately in, for instance, the patient's mouth. A prosthesis may then be constructed and the implant loaded immediately. This method is less popular because immediate loading carries with it an increased risk of failure as the implant may not be sufficiently stable to distribute the stresses from the prosthesis effectively. In a two stage procedure, the implant is placed in two parts and the implant fixture is buried beneath the soft tissue and left to heal for three to six months before connection of a metal collar or transmucosal abutment. This transmucosal abutment then allows connection of the prostheses. It is generally accepted that the success of a two stage procedure is higher because of the delayed loading.

The key to successful implant placement is achievement of good implant stability. Implant stability is the resistance of an implant to movement and reflects its ability to distribute stresses. The stability of an implant at placement is a function of a number of parameters. These relate to the implant itself, its length, diameter, and surface characteristics. They also relate to the type of surgical procedure, the size of hole that is drilled and the amount of tissue removed. Equally important is the quality of the bone, which may vary from a dense cortical plate in the anterior part of the mandible to an open trabecular network in the posterior part of the maxilla. Following implant placement stability chances as a healing and remodeling process takes place within the bone. It is likely that there is a degree of stress relaxation following the placement of the implant followed by an inflammatory response with wound healing. Following this there will be remodeling until an equilibrium stage is reached. A successful, osseointegrated implant shows no decrease in the height of bone surrounding it nor a decrease in stiffness. The current most commonly used method for assessing implant performance is the use of radiographs, however these are two dimensional in nature and difficult to reproduce. It has been demonstrated that implant stability and bone height can be related to the first (and higher) resonance frequencies of a transducer attached to the implant. It has also been demonstrated that the resonance frequency could be measured on the implant itself.

A measurement of implant stability is a useful parameter for both the diagnosis of problem implants and the monitoring of implants throughout their lifetime. It is generally referred to in WO92/18053, U.S. Pat. No. 5,518,008, U.S. Pat. No. 5,392,779 and U.S. Pat. No. 4,499,906. Through the prior art it is known to use the resonance frequency as a parameter, but this causes problems when the resonance is used for determining the stability and is affected by another factors than the stability.

The basic parameter for the measurement of implant stability is resonance frequency (Fr). Fr is specific for an implant situation and, as described above, is dependent on a number of different parameters, for instance the geometry and the material of the implant. This means that measurements on different implants can give different resonance frequencies although they have the same stability. In practice, this makes it difficult to evaluate the real stability in the actual case. It is one objective of the invention to solve this problem and make it possible to determine the true stability in spite of differences between different implants.

When the measurement of implant stability is made indirectly, by measuring the resonance frequency with a transmucosal abutment or other structure mounted on the implant, the geometry of the abutment and/or structure will affect the resulting resonance frequency. This will make it difficult to directly compare measurements of Fr for objects in different situations.

It is a general objective of the invention to solve this problem.

The resonance frequency can have a substantial variation, which means that an instrument that can cover a wide frequency range is necessary. In normal situations, the clinically interesting range is smaller than what is actually possible, leading to difficulties in achieving good resolution in that range. It is an objective of the invention to solve this problem.

For a stability value to be useful to a clinician, the presented value has to be comparable between implants of different types, different lengths and also between situations with different clinical/surgical conditions (for instance the drill hole diameter, the bone quality and the anatomical area).

It is an objective of the invention to solve this problem.

It is of interest to compare stability measurements made at different occasions during the treatment process. It is an objective of the invention to solve this problem.

That mainly can be considered as characteristic for a device according to the invention is, amongst other things, that it contains receiving organs which receives the respective frequency signal and transforms it to a digital information signal which is related to and/or is representing the stability and/or, under the above specific circumstances, distinctive features in the implant situation. Further characteristics are the it works together with organs that contains information which shows or is working with information which represents those distinctive features and that it also contains a calculation unit (for example a microcomputer), or is working together with such a unit which with a software program processes one or more information signal(-s) with the above information. According to the invention, the processing with a preferably mainly known program, results in a presentation information, which could be transferred to a presentation unit which could be separate or included in the calculation unit and arranged to present the stability independent. of the implant type, geometry et cetera. It is also referred to the characteristic parts of claims 1, 2 and 3.

The invention proposes i.a. the use of internal memory circuits and/or an external memory unit and/or memory circuits in the transducer. The memory circuits contains in use information about the present implant system, i.e. the distinctive features of the implant of the patient to be assessed. The memory contents is used in an arithmetic unit (for example a microcomputer), which can be built in the instrument or be external. The arithmetic unit uses the information in the memory circuits to compensate the measurement value for differences in the geometry and other factors between different implant systems.

In one embodiment where, for example, the implant is used with an abutment, information about the dimensions of the specific abutment and/or other specific information are used in the same way to compensate the measurement value for influences from these. In another embodiment, the system of the invention uses knowledge about which is the clinically interesting frequency range, to map Fr to a scale with good resolution in that range. The scale could run from, for instance 0 to 100.

These previous versions have the form of a system or device, which translates Fr to a compensated value, given the abutment, dimensions (if an abutment is used) and information about the implant system used, as input. It could also be used to compensate for other clinical parameters, such as the diameter of the drilled hole, the quality of the bone, the anatomical area et cetera, which can be added by the clinician or otherwise communicated to the arithmetic unit. In a further aspect of the present invention there is provides a device as claimed in claim 9 of the claims hereinafter. The device the mentioned receiving organs contains or cooperates with one or more storage units, internal and/or external, which stores the digital information signal, and/or its presentation, for each respective measurement, such that the signal(-s) and/or presentation(-s) is/are reusable in order to be run together or compared with other corresponding information signals and/or presentations that are received at consecutive interactions, separated in time, between the implant(-s) and one or more vibration effectuating unit. It is also referred to the characteristic part of claim 9. According to a yet further aspect of the present invention there is provided a method as claimed in claim 12 of the claims hereinafter.

In one version, stability values are stored in the instrument or in external memory circuits, to make it possible to compare measurements made at different occasions, and simplify for the clinician and/or let the arithmetic unit make calculations based upon these comparisons.

All suggested solutions are achieved by using hardware to determine the resonance frequency in a specific frequency range, and then using an arithmetic unit together with stored information for determining the stability value.

A working set-up and method according to the invention may look and work according to the attached drawing, which partly and principally shows a longitudinally sectioned view of an implant in bone, and an instrument, in a block diagram, for measuring implant stability.

An embodiment of the present invention will now be more particularly described by way of example with references to the accompanying drawing, which principally discloses components and signals included and attained in the actual apparatus. The drawing shows a bone K. The implant 1 has been applied in the bone according to known procedures, and can be selected from a range of diameters D and lengths L. A vibration-or force effectuating and vibration detecting unit (1) is brought in contact with the implant (or an attached structure). The unit is connected or possible to connect to an instrument In, through a connection 1. ed. The instrument includes vibration effectuating unit (2), an A/D-converter (3), an arithmetic unit (microcomputer) (4), one or more memories (5, 6), a presentation unit (7), a memory for storing measurement data (8), and a comparing unit (9).

A frequency signal io is sent from the unit, and a frequency signal is achieved from the unit (1) when it is activated against the implant (or attached structure). The unit (3) converts the signal i₁ to a digital information signal i₂. The unit (4) which can work with a known program, processes the signal i₂ combining it with the signal i₃ which is representative for distortion factors such as differences in diameter and length (D and L), and more. The result is a presentation signal i₄ which relates only to the stability and not to any of the distortion factors.

The program executes the equation ISQ=F×(F,−Fl)/(Fh−F,)×100, where ISQ=implants stability.

F=a compensating function which can be linear or non-linear and dependent of the implant geometry; the geometry of the abutment or any other attached structure, clinical conditions et cetera.

F,=low limit of the clinically relevant frequency range. Fr=the resonance frequency.

Fh=high limit of the clinically relevant frequency range F, preferably has values around 5000 Hz (4500-5500 Hz), and Fₕpreferably has values around 10000 Hz (9000-11000 Hz).

In a case in which one would use measurements separated in time or measure the stability at two or more occasions, the memory (8) and the comparator (9) is used. Storage of measurements and/or reusable values is indicated with i;, and the comparing function with i₆. The program in the unit (4) is illustrated as the memory function (6). Also, activating buttons or a keyboard is indicated (10). Control information i₇ can be typed in or received with the keyboard. In the example, the functional components 2-10 are realized in the instrument In. One or more of the components can be external components to which the instrument is attachable at or between the measurement occasions. The instrument can interact with other equipment (for example computer equipment, communication wires, computer network et cetera) which is symbolized with (11) and its connections with Led, at which signal is present.

In an embodiment sensors are used. Each model of sensor is then applied on several calibrating block having a known stability and in which a function is decided by means of a resonance frequency as a function of the stability (defined as ISQ, scaled with 0-100). The factors of this function are programmed in a memory applied in the connecting member of the sensor, together with an individual calibrating factor for each sensor (this one is decided on one or more calibrating blocks after the manufacture of the sensor). If the sensor is to be adapted on distance level, the procedure is the same, apart from the fact that the resonance frequency is measured for several different lengths of distances on respective calibrating block. Factors, which compensate for the distance length, are also memorized in the memory of the connection member.

In another embodiment only the resonance frequency is shown and after that the sensor is used on a calibrating block with known stability is calibrated. The absolute stability then can be calculated.

In a general embodiment the new apparatus and method can be used for checking the stability of a unit (first unit) which is enclosed in a substrate, foundation, material etc. in a corresponding way as above with implant and bore. The first unit may be mounted and/or is preformed with some elasticity, spring effects, etc. The preambles, characteristic part of the independent claims and the sub-claims can be mutually.

The invention is not limited to the above example, and can undergo modifications within the following claims and the intention of the invention.

What is claimed is:

1. Device for determining the stability of an implant fitted in the human body, preferably in the facial skeleton, where a vibration-producing unit cooperates with the implant and gives a frequency signal (i1) which is a function of its stability and characteristic features, e.g., size, position, etc., characterized in that it operates with a digital information signal (i2) which represents said stability and feature and is generated in a receiver unit which receivers the frequency signal (i1) and converts it to said digital information signal (i2), in that the information item(s) representing the characteristic features are included in or connected to information-containing elements (memories) included in or connected to the device, and in that one or more information signals (i4) principally represent only the stability, which information signal or signals (i4) result(s) from the processing, in an arithmetic unit, preferably in a microprocessor, belonging to or connected to the device, of the digital information signal (i2) and the information item(s) representing the characteristic features in order to permit information relating only to the stability to be displayed in a display unit.

2. Device according to claim 1, characterized in that it is arranged with an integrated unit or can be operated together with a separate unit, for example said processor unit or a second processor unit, by means of which respective display results or storage results can be stored so as to be able to be compared with one or more preceding display or storage results.

3. Device according to claim 1 or 2, characterized in that the calculation of the stability can be defined by the equation ISQ=F×(Fᵣ−Fₗ)/(Fₕ−Fₗ)×100, where ISQ designates the implant stability, F designates a compensating function which can be linear or nonlinear and dependent on the geometry of the implant, the geometry of the spacer or other attachment structure, the clinical conditions, etc., Fₗ designates the lower limit for the clinically relevant measurement range, Fᵣ designates the resonance frequency, and Fₕ designates the upper limit of the clinically relevant measurement range.

4. Device according to claim 1 or 2, characterized in that Fₗ has a value of approximately 15000 Hz

5. Device according to claim 3, characterized in that Fₗ has a value of approximately 5000 Hz

6. Device according to claim 1 or 2, characterized in that Fₕ has a value of approximately 10000 Hz.

7. Device according to claim 3, characterized in that Fₕ has a value of approximately 10000 Hz.

8. Device according to claim 4, characterized in that Fₕ has a value of approximately 10000 Hz.

9. Device according to claim 5, characterized in that Fₕ has a value of approximately 10000 Hz.

10. Device according to claim 1 or 2, characterized in that F is a nonlinear function.

11. Device according to claim 3, characterized in that F is a nonlinear function.

12. Device according to claim 4, characterized in that F is a nonlinear function.

13. Device according to claim 5, characterized in that F is a nonlinear function.

14. Device according to claim 6, characterized in that F is a nonlinear function.

15. Device according to claim 7, characterized in that F is a nonlinear function.

16. Device according to claim 8, characterized in that F is a nonlinear function.

17. Device according to claim 9, characterized in that F is a nonlinear function.

18. Device for determining t he stability of an implant fitted in the human body, preferably in the facial skeleton, where a vibration-producing unit cooperates with the implant and gives a frequency signal (i1) which is a function of its stability, characterized in that it operates with a digital information signal which represents or is related to the implant's stability, which digital information signal (i2) is generated in a receiver unit which converts the frequency signal to said digital information signal, in that the digital information signal or the display relating to the respective implant is stored in one or more storage units (memories) included in or connected to the device, and in that the digital information signal(s) thus stored for the respective implant can be recovered for coordination or comparison with a further digital information signal or display which occurs in the event of a time-lagged subsequent cooperation between the implant and the vibration-producing unit.

19. Device according to claim 18, characterized in that first and second storage units (memories) store information signal(s/display(s)) from two or more readings or test cases, in that comparison elements are provided to compare information signals and/or displays supplied from the first and second storage units and to send to the display element concerned a comparison information item which is a function of the comparison and which represents actual stability during a time period extending from approximately one month and onward.

20. Device according to claim 18 or 19 characterized in that it emits an information item or display corresponding to the actual stability and irrespective of the implant's length of contact in the jaw or bone, diameter, volume, etc.

21. A method to measure the stability of the anchorage of a unit, here called first unit, which is anchored in a substrate (material), where one or more vibration effectuating units is applied to the anchored unit or an attached structure and where one or more stability dependent frequency signals is achieved from the vibration effecting unit(-s), characterized in:
a) that the frequency signal(-s) is/are converted to one or more information signal(-s) representing the stability,
b) that the information signal(-s) is/are processed in a processor means to eliminate distortion factors dependent of the structure of the first unit, implement situation etc. to provide information output signal that is dependent mainly only on the stability, and
c) that the last mentioned information output signal is transferred to direct use in said measure and/or is stored in one or more memory means to be reused for comparison between two or more set of presentation information, separated in time

22. A method according to claim 21, characterized in that it detects the stability of an implant in the human body, preferably in the facial skeleton.
